(19) United States
(12) Patent Application Publication
(10) Pub. No.: US 2023/0317275 A1
(43) Pub. Date: Oct. 5, 2023

(54) VIRTUAL AND AUGMENTED REALITY FOR TELEHEALTH

(71) Applicant: Welch Allyn, Inc., Skaneateles Falls, NY (US)

(72) Inventors: Gene J. Wolfe, Pittsford, NY (US); Patrice Etchison, Cary, NC (US); Craig M. Meyerson, Syracuse, NY (US); Daniel Shirley, Raleigh, NC (US); Carlos Andres Suarez, Syracuse, NY (US)

(21) Appl. No.: 18/185,454

(22) Filed: Mar. 17, 2023

Related U.S. Application Data
(60) Provisional application No. 63/327,088, filed on Apr. 4, 2022.

(51) Int. Cl. G16H 40/67; G06T 19/00; H04N 7/15; G06T 7/62; G06F 3/01; G06F 3/16; G16H 80/00

(52) U.S. Cl. CPC G16H 40/67; G06T 19/006; H04N 7/15; G06T 7/62; G06F 3/017; G06F 3/167; G16H 80/00; G06T 2207/10024

(57) ABSTRACT

A device for conducting a telehealth consultation. The device receives a video feed of a patient environment, and recognizes a local device in the video feed of the patient environment. The device detects an input directed to the local device, generates a control command based on the input, and sends the control command to the local device. The control command causes the local device to perform an action during the telehealth consultation.

### BACKGROUND

Successful patient outcomes often correlate with a level of patient engagement. For example, patients who are more engaged with caregivers tend to have higher compliance with their treatment and more successful outcomes. Augmenting engagement between patients and caregivers can thus improve healthcare quality and outcomes.

Also, contemporary video conferencing systems are not optimized for telehealth services because interactivity between remote caregivers and patients is limited. Another problem is that remote caregivers are not physically in the room to interact with the patient or with the monitoring and therapeutic devices attached to the patient. These obstacles may cause reluctance for patients and healthcare providers to use telehealth systems.

### SUMMARY

In general terms, the present disclosure relates to telehealth. In one possible configuration, a telehealth device provides an improved interaction between a patient and a remote caregiver during a telehealth consultation. Various aspects are described in this disclosure, which include, but are not limited to, the following aspects.

One aspect relates to a device for conducting a telehealth consultation, the device comprising: at least one processing device; and a memory device storing instructions which, when executed by the at least one processing device, cause the at least one processing device to: receive a video feed of a patient environment; recognize a local device in the video feed of the patient environment; detect an input directed to the local device; generate a control command based on the input; and send the control command to the local device, wherein the control command causes the local device to perform an action during the telehealth consultation.

Another aspect relates to a method of conducting a telehealth consultation, the method comprising: receiving a video feed of a patient environment; recognizing a local device in the video feed of the patient environment; detecting an input directed to the local device, the input detected from a remote caregiver watching the video feed; generating a control command based on the input; and sending the control command to the local device, wherein the control command causes the local device to perform an action during the telehealth consultation.

Another aspect relates to a device for conducting a telehealth consultation between a patient and a remote caregiver, the device comprising: at least one processing device; and a memory device storing instructions which, when executed by the at least one processing device, cause the at least one processing device to: receive a video feed of the patient; display a three-dimensional image based on the video feed; determine a measurement of a feature in the three-dimensional image; and display the measurement in the three-dimensional image.

Another aspect relates to a method of conducting a telehealth consultation, the method comprising: receiving a video feed of a patient, the video feed including red, green, blue, and depth (RGB-D) data; displaying a three-dimensional image of the patient based on the RGB-D data; determining a measurement of a feature based on the RGB-D data; and displaying the measurement in the three-dimensional image.

### DESCRIPTION OF THE FIGURES

The following drawing figures, which form a part of this application, are illustrative of the described technology and are not meant to limit the scope of the disclosure in any manner.

FIG. 1 illustrates an example of a virtual care system that can provide virtual and/or augmented reality for telehealth services.

FIG. 2 illustrates an example of a patient telehealth device that is included in the virtual care system of FIG. 1.

FIG. 3 schematically illustrates an example of the patient telehealth device of FIG. 2.

FIG. 4 schematically illustrates a range of viewing angles for viewing aspects of an image displayed by an image forming device of the patient telehealth device of FIG. 2.

FIG. 5 is an isometric view of an example of the patient telehealth device of FIG. 2.

FIG. 6 schematically illustrates an example of a remote caregiver telehealth device that is included in the virtual care system of FIG. 1.

FIG. 7 schematically illustrates an example of a method of conducting a telehealth consultation by the virtual care system of FIG. 1.

FIG. 8 schematically illustrates another example of a method of conducting a telehealth consultation by the virtual care system of FIG. 1.

### DETAILED DESCRIPTION

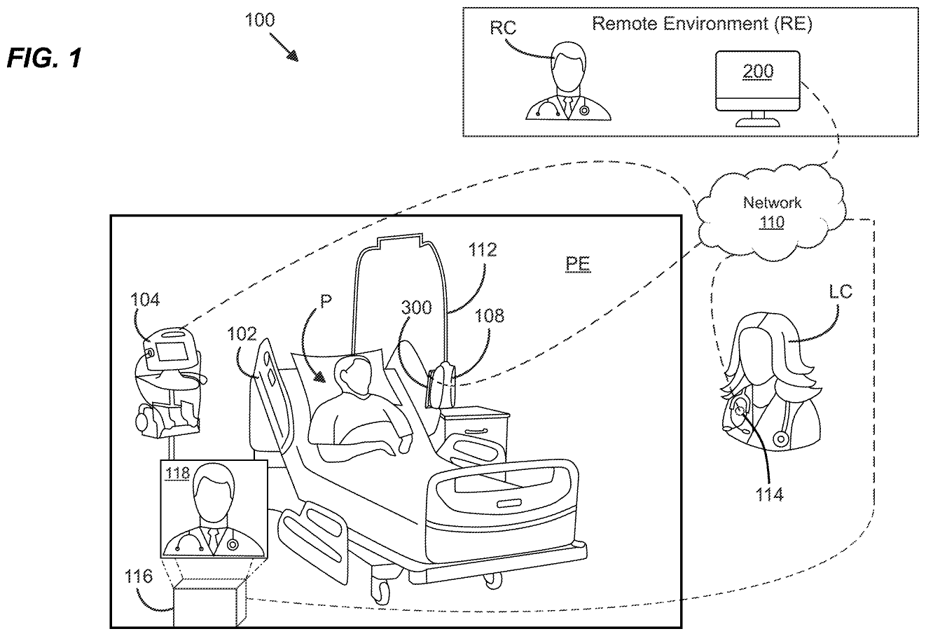

FIG. 1 illustrates an example of a virtual care system 100 that can improve engagement and interaction between a patient P located in a patient environment PE and a remote caregiver RC located in a remote environment RE. The virtual care system 100 can be used to provide telehealth services. As described herein, telehealth is the distribution of health-related services and information via electronic information and telecommunication technologies.

In the example shown in FIG. 1, the virtual care system 100 includes a remote caregiver telehealth device 200 that is operable by the remote caregiver RC in the remote environment RE. The remote caregiver telehealth device 200 is in communication with various devices located in the patient environment PE via a communications network 110. For example, the remote caregiver telehealth device 200 is in communication with at least a patient support apparatus 102, a vital signs monitoring device 104, a camera 114, a projector device 116, and a patient telehealth device 300. These devices will each be described in more detail below.

The remote environment RE is remotely located with respect to the patient environment PE. In one example, the patient environment PE is a patient room within a healthcare facility such as a hospital, a nursing home, a long term care facility, and the like. In such examples, the remote environment RE is a different room or location within the healthcare facility. In further examples, the remote environment RE is offsite such as a separate building, facility, campus, or other remote site location.

In another example, the patient environment PE is the home of the patient P. In such examples, the remote environment RE is a healthcare facility such as a hospital. In such examples, the virtual care system 100 can be used to provide hospital-at-home healthcare services. The hospital-at-home healthcare services enable the patient P to receive acute-level care in their home, rather than in an acute care setting such as a hospital or other healthcare facility.

The communications network 110 can include any type of wired or wireless connections or any combinations thereof. The communications network 110 includes the Internet. In some examples, the communications network 110 includes wireless connections such as cellular network connections including 4G or 5G. Wireless connections can also be accomplished using Wi-Fi, ultra-wideband (UWB), Bluetooth, and the like.

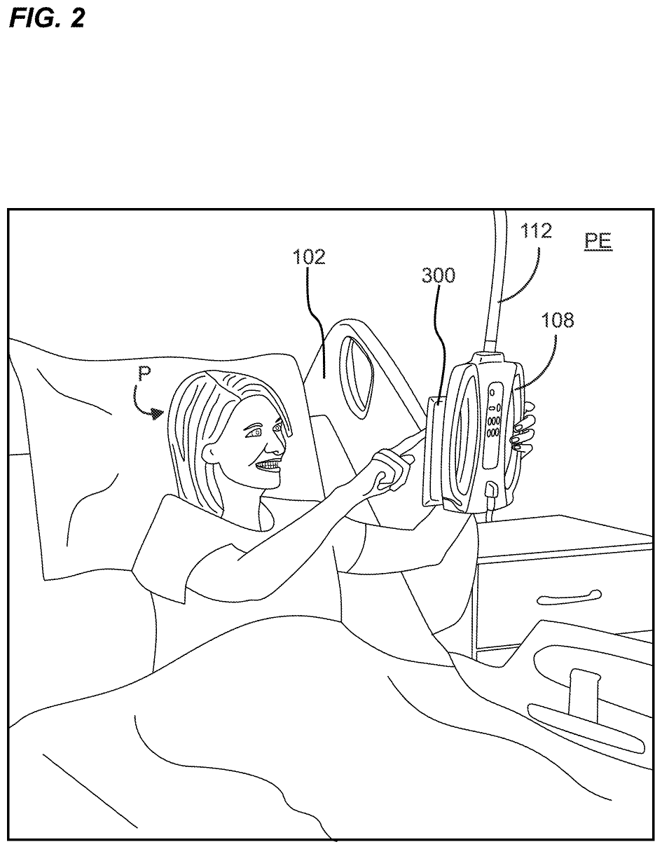

FIG. 2 illustrates another view of the patient telehealth device 300. Referring now to FIGS. 1 and 2, the patient support apparatus 102 is depicted as a hospital bed on which the patient P rests. The patient support apparatus 102 can include an overhead arm assembly 112 that supports the patient telehealth device 300 in front of the patient P. The patient telehealth device 300 can removably attach to a support structure 108 connected to a distal end of the overhead arm assembly 112. The overhead arm assembly 112 and the support structure 108 can support a variety of devices having different shapes and sizes such as tablet computers, smartphones, laptops, patient pendants for controlling operation of the patient support apparatus 102, and other electronic devices. In some examples, the patient support apparatus 102, the overhead arm assembly 112, and the support structure 108 share similarities with the devices described in U.S. Pat. No. 11,103,398, which is incorporated by reference in its entirety.

In the example shown in FIGS. 1 and 2, the patient telehealth device 300 is designed to have an appropriate size for easy manipulation and handling by the patient P while seated or in a supine position in the patient support apparatus 102. As an example, the patient telehealth device 300 can have a shape and size resembling that of a tablet computer.

The vital signs monitoring device 104 can include or otherwise be connected to one or more physiological sensors for measuring and recording physiological parameters of the patient P. Examples of the physiological sensors can include sensors for measuring and recording pulse rate, blood oxygen saturation (SpO2), non-invasive blood pressure (systolic and diastolic), respiration rate, temperature, electrocardiogram (ECG), heart rate variability, and the like. In some examples, the vital signs monitoring device 104 is a spot monitor, similar to the one described in U.S. Pat. No. 9,265,429, which is incorporated by reference in its entirety.

Referring now to FIG. 1, the camera 114 is worn by a local caregiver LC who is in the patient environment PE. The camera 114 captures a video feed from the perspective or viewpoint of the local caregiver LC, and transmits the video feed for display on the remote caregiver telehealth device 200. The camera 114 can communicate the video feed to the remote caregiver telehealth device 200 via the communications network 110.

In the example shown in FIG. 1, the camera 114 is depicted as a body worn camera such as a camera that can attach to an article of clothing worn by the local caregiver LC. In further examples, the camera 114 can be integrated into eyeglasses worn by the local caregiver LC. In yet further examples, the camera 114 can be integrated into a headband or headset worn by the local caregiver LC. Additional examples for where or how to mount the camera 114 to provide a video feed from the viewpoint of the local caregiver LC are contemplated.

As further shown in FIG. 1, the projector device 116 is positioned inside the patient environment PE. The projector device 116 can receive a video stream of the remote caregiver RC from the remote caregiver telehealth device 200 via the communications network 110. The projector device 116 uses the video stream of the remote caregiver RC to generate and display a holographic image 118 of the remote caregiver RC inside the patient environment PE. The holographic image 118 can improve engagement and interaction between the patient P located in the patient environment PE and the remote caregiver RC located in the remote environment RE.

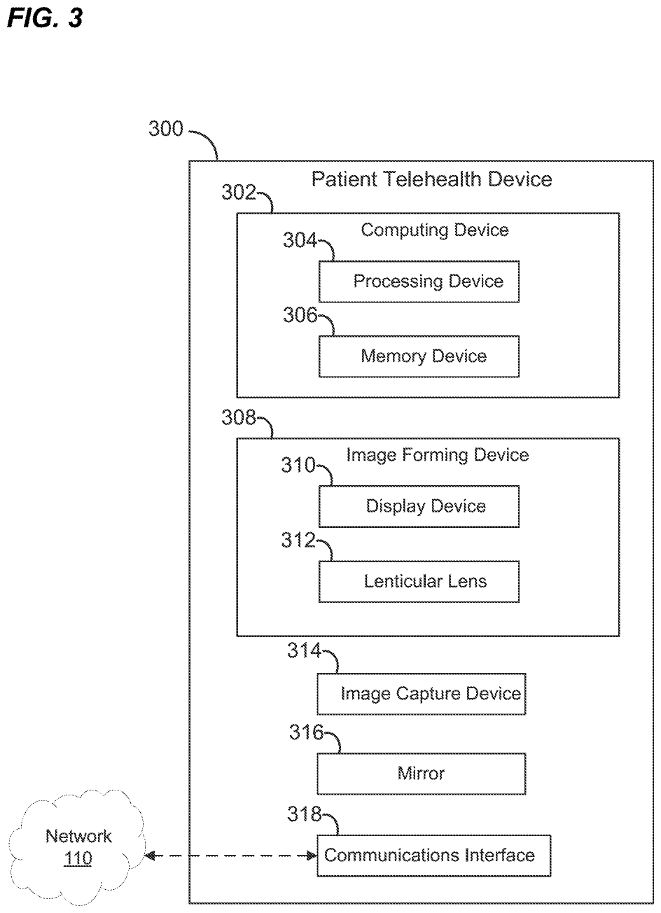

FIG. 3 schematically illustrates an example of the patient telehealth device 300. As shown in FIG. 3, the patient telehealth device 300 includes a computing device 302 having a processing device 304 and a memory device 306. The computing device 302 enables capture, encoding, processing, compression, transmission, decompression, and rendering of red, green, blue, and depth (RGB-D) data by the patient telehealth device 300.

The processing device 304 is an example of a processing unit such as a central processing unit (CPU). The processing device 304 can include one or more CPUs. The processing device 304 can further include one or more microcontrollers, digital signal processors, field-programmable gate arrays, and other electronic circuits.

The memory device 306 stores data and instructions for execution by the processing device 304. The memory device 306 includes computer-readable media, which may include any media that can be accessed by the patient telehealth device 300. The computer-readable media can include computer readable storage media and computer readable communication media.

The computer readable storage media can include volatile and nonvolatile, removable and non-removable media implemented in any device configured to store information such as computer readable instructions, data structures, program modules, and other data. The computer readable storage media can include, but is not limited to, random access memory, read only memory, electrically erasable programmable read only memory, flash memory, and other types of memory technology, including any medium that can be used to store information. The computer readable storage media is non-transitory.

The computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, the computer readable communication media includes wired media such as a wired network, and wireless media such as acoustic, radio frequency, infrared, and other wireless media. Combinations of any of the above are within the scope of computer readable media.

The patient telehealth device 300 includes an image forming device 308 that can improve engagement and interaction between the patient P and the remote caregiver RC by providing a three-dimensional (3D) image that allows the patient P's to view different aspects of the 3D image when the patient views the 3D image from different viewing angles. As will be described in more detail, the 3D image is generated by a light field lenticular multi angle display.

The image forming device 308 includes a display device 310 that operates to display an image or a video stream. In some instances, the display device 310 displays an image or video stream of the remote caregiver RC captured by the remote caregiver telehealth device 200. In some instances, the display device 310 displays an image or video stream of a diagnostic image taken of the patient P for the patient P to better understand anatomical structures shown in the diagnostic image, and to thereby better understand a diagnosis provided by the remote caregiver RC. In some examples, the display device 310 includes a liquid-crystal display (LCD) that uses light-modulating properties of liquid crystals and a backlight to produce images.

The image forming device 308 further includes a lenticular lens 312 that allows the patient P to see different aspects of the images displayed by the display device 310 such as when the patient P changes their viewing angle of the display device 310 either due to the patient P moving relative to the display device 310, or the patient P moving the patient telehealth device 300. The lenticular lens 312 can include an array of lenses positioned in front of the display device 310 to generate a 3D rendering based on the images displayed by the display device 310.

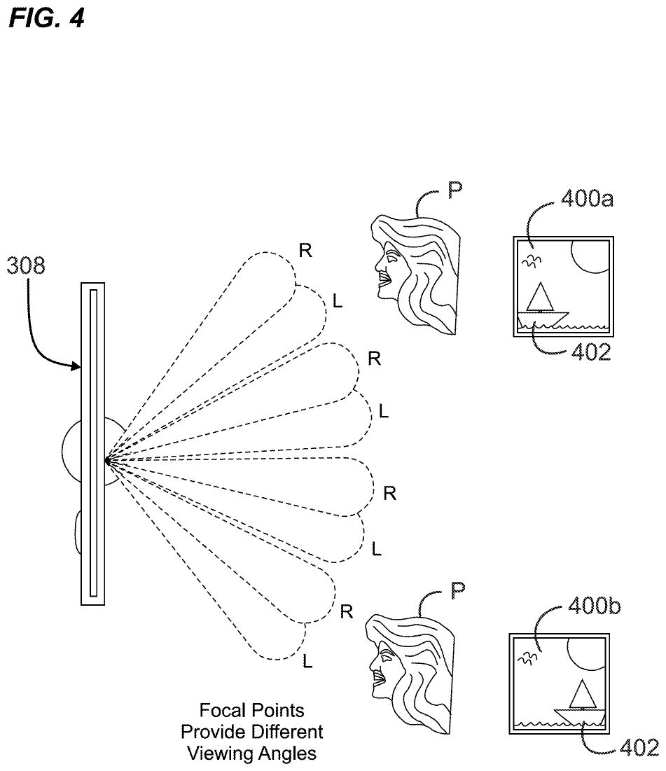

FIG. 4 schematically illustrates an example of the image forming device 308 showing a range of viewing angles for the patient P to view different aspects of an image displayed by the image forming device 308. The image forming device 308 can provide depth perception and allow the patient P to perceive an image from different perspectives or viewing angles. As the patient P adjusts their viewing angle, the image forming device 308 allows the patient P to see different pixels displayed by the display device 310 behind the lenticular lens 312.

As an illustrative example, when the patient P views the image forming device 308 at a right-most angle, an object 402 in an image 400a appears toward a left side of the image. As patient P changes their viewing angle from right to left, the position of the object 402 in the image changes. For example, when the patient P views the image forming device 308 at a left-most angle, the object 402 in an image 400b appears toward a right side of the image. This allows the patient P to view different aspects of an image with depth perception at multiple viewing angles with respect to the patient telehealth device 300 without the need for specialized eyewear accessories for viewing 3D images such as goggles, eyeglasses, and the like.

This can improve the engagement and interaction between the patient P and the remote caregiver RC. For example, the remote caregiver RC during a telehealth consultation with the patient P can use the remote caregiver telehealth device 200 to cause the display device 310 to display a diagnostic image of the patient P such as an image of the patient's heart. During the telehealth consultation, the patient P can adjust their viewing angle of the patient telehealth device 300 such as by moving their eyes relative to the display device 310, or moving the patient telehealth device 300 relative to their eyes, to view different aspects of the diagnostic image to have a better understanding of a diagnosis provided by the remote caregiver RC. This can increase the patient P's engagement during the telehealth consultation with the remote caregiver.

Referring back to FIG. 3, the patient telehealth device 300 further includes an image capture device 314 that operates to capture an image or video feed of the patient P while the patient is using the patient telehealth device 300. The image capture device 314 allows for two-way video communications between the patient P and the remote caregiver RC. For example, the patient telehealth device 300 can transmit the image or video feed captured by the image capture device 314 to the remote caregiver telehealth device 200 via the communications network 110.

The image capture device 314 includes a red, green, and blue color (RGB) image sensor and a depth sensor, also known as an RGB-D camera, which captures images augmented with depth information (related with the distance to the sensor) in a per-pixel basis.

The patient telehealth device 300 further includes a mirror 316 that is coupled to or otherwise covers the image capture device 314. The mirror 316 is a half mirror or two-way mirror that allows the image capture device 314 to capture images without being seen by the patient P such that the image capture device 314 does not disturb the 3D image generated by the display device 310 and the lenticular lens 312. For example, the mirror 316 is reflective on a side facing the patient P such that the patient P cannot see the image capture device 314 when viewing the 3D image displayed by the display device 310 and the lenticular lens 312. The mirror 316 is transparent on an opposite side facing the image capture device 314 such that the image capture device 314 is able to capture an image or video stream of the patient P.

In some alternative examples, the patient telehealth device 300 includes a neutral density area of the image forming device 308. The neutral density area is constructed such that the image capture device 314 is able to image through the image forming device 308 such that the vanishing point of a field of view is centrally located within the image forming device 308.

The patient telehealth device 300 further includes a communications interface 318 that operates to connect the patient telehealth device 300 to the communications network 110 for communication with the remote caregiver telehealth device 200. The communications interface 318 can include both wired interfaces (e.g., USB ports, and the like) and wireless interfaces (e.g., Bluetooth, Wi-Fi, ultra-wideband, and similar types of wireless protocols).

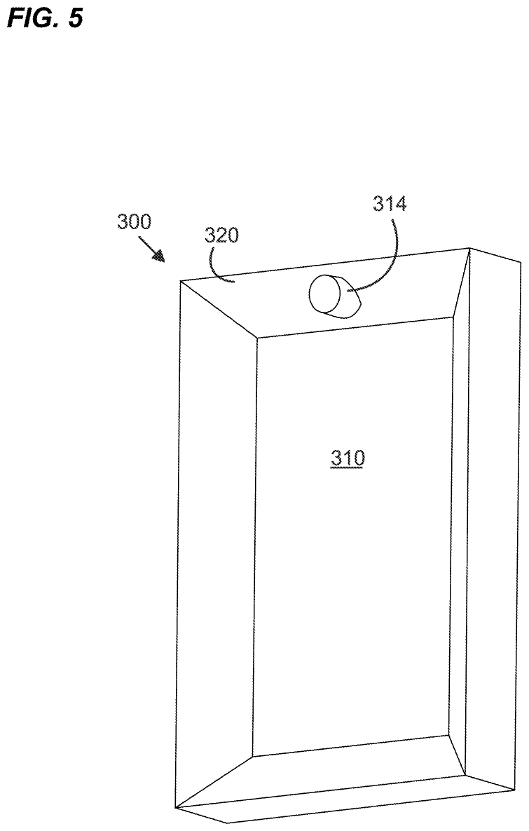

FIG. 5 is an isometric view of the patient telehealth device 300. As shown in FIG. 5, the patient telehealth device 300 includes a debossed bezzle 320 that surrounds the display device 310. The debossed bezzle 320 provides a spatial isolation reference platform around the 3D visualization provided by the display device 310 and the lenticular lens 312.

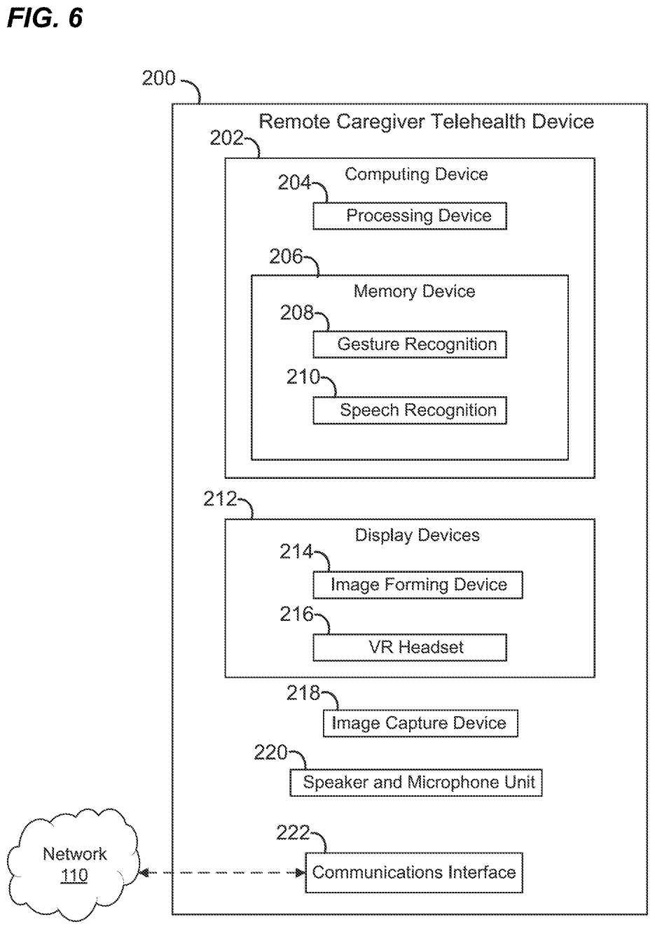

FIG. 6 schematically illustrates an example of the remote caregiver telehealth device 200. The remote caregiver telehealth device 200 shares many similar components with the patient telehealth device 300. For example, the remote caregiver telehealth device 200 includes a computing device 202 having a processing device 204 and a memory device 206, an image forming device 214, an image capture device 218, and a communications interface 222 that can be substantially similar or the same as the computing device 302, the processing device 304, the memory device 306, the image forming device 308, the image capture device 314, and the communications interface 318 of the patient telehealth device 300, described above.

The memory device 206 stores one or more gesture recognition algorithms 208 and speech recognition algorithms 210, which allow the remote caregiver RC to control one or more devices in the patient environment PE using gestures and/or voice. The gesture recognition algorithms 208 and the speech recognition algorithms 210 will be described in more detail.

As shown in FIG. 6, the remote caregiver telehealth device 200 includes one or more display devices 212 which can include the image forming device 214. In some examples, the image forming device 214 includes the same components as the image forming device 308 of the patient telehealth device 300 such as a display device and a lenticular lens that can generate a 3D image allowing the remote caregiver RC to view different aspects of the 3D image when the remote caregiver RC views the 3D image from different viewing angles. In such examples, the remote caregiver telehealth device 200 and the patient telehealth device 300 are symmetrical in that they share the same components that provide the same functionality.

In alternative examples, the remote caregiver telehealth device 200 and the patient telehealth device 300 are asymmetrical in that they include different components that provide different functionalities between the two devices. For example, in one embodiment, the remote caregiver telehealth device 200 generates 3D images, while the patient telehealth device 300 generates 2D images. In another embodiment, the remote caregiver telehealth device 200 generates 2D images, while the patient telehealth device 300 generates 3D images.

In examples where the image forming device 214 generates a 3D image, the remote caregiver telehealth device 200 allows the remote caregiver RC to view details of the patient that are not ordinarily viewable on conventional 2D display monitors. For example, the 3D image can provide enhanced imaging of the patient for viewing details such as facial droop that may not be easily seen or detected by the remote caregiver RC when viewing standard 2D images.

Additionally, the 3D image can provide enhanced imaging of a wound on the patient P such as by providing depth perception. In some examples, the red, green, blue, and depth (RGB-D) data allows the processing device 204 to more accurately and efficiently calculate a size and depth measurement of the wound. For example, the processing device 204 can measure the size and depth of the wound without having to perform complex spatial calibrations, photometric interpretation, and other imaging techniques that rely on pixel counts.

The 3D image of the patient P displayed on the one or more display devices 212 can be augmented to display clinical information recorded from the patient environment PE such as physiological measurements from the vital signs monitoring device 104 such as pulse rate, blood oxygen saturation (SpO2), non-invasive blood pressure (both systolic and diastolic), respiration rate, temperature, electrocardiogram (ECG), heart rate variability, and the like.

Also, the 3D image of the patient P displayed on the one or more display devices 212 can be augmented to display additional clinical data such as measurements captured by a stethoscope, otoscope, thermometer, or other instrument operated by the local caregiver LC. The clinical data from the instrument (including video and diagnostic measurements and values) can be streamed to the remote caregiver telehealth device 200 via the communications network 110.

Also, the 3D image of the patient P displayed on the one or more display devices 212 can be augmented to display measurements calculated based on the RGB-D data. For example, the size, depth, shape, and color of wounds and other features on the patient P can be calculated based on the RGB-D data and can be displayed together with the 3D image of the patient.

The clinical information including physiological measurements from the vital signs monitoring device 104, video and diagnostic measurements from handheld devices operated by the local caregiver LC, and measurements calculated based on the RGB-D data can be overlayed the 3D image to improve data presentation provided by the remote caregiver telehealth device 200 for consumption by the remote caregiver during a telehealth consultation with the patient.

In some further examples, the remote caregiver telehealth device 200 can automatically detect and/or identify the devices present in the patient environment PE and/or the devices connected to the patient P. For example, the identification of the devices present in the patient environment PE and/or connected to the patient P can be based on the video feed captured by the camera 114 worn by the local caregiver LC, or by a video feed captured by another camera in the patient environment PE such as a pan, tilt, zoom (PTZ) camera that can provide a 360 degree view of the patient environment PE. During a telehealth consultation between the remote caregiver RC and the patient P, the remote caregiver telehealth device 200 can automatically display relevant data from the identified devices, or can provide controls for the remote clinician RC to control the identified devices during a telehealth consultation.

As further shown in FIG. 6, the one or more display devices 212 can further include a virtual reality (VR) headset 216 that can display a video feed captured by the camera 114 worn by the local caregiver LC (see FIG. 1). This allows the remote caregiver RC to view the patient P and the patient environment PE from the perspective of the local caregiver LC. In some examples, the video feed captured by the camera 114 can be displayed on the image forming device 214 or on a standard 2D monitor for viewing by the remote caregiver RC.

The remote caregiver RC can instruct the local caregiver LC using a speaker and microphone unit 220 to view a particular object in the patient environment PE such as the patient P, or a device in the patient environment PE such as the patient support apparatus 102 or the vital signs monitoring device 104. Once the local caregiver LC moves closer to the object, the camera 114 can capture a video feed of the object. In some examples, the computing device 202 can automatically recognize the object based on the video feed received from the camera 114.

In examples where the object is a device in the patient environment PE, the computing device 202 can automatically recognize the device such as the patient support apparatus 102 or the vital signs monitoring device 104. Thereafter, the computing device 202 can provide a virtual interface for the remote caregiver RC to remotely control the device. The virtual interface can be displayed on the one or more display devices 212 such as on the image forming device 214, the VR headset 216, or a standard 2D monitor for viewing by the remote caregiver.

In some examples, the virtual interface enables control of the device using gestures including hand gestures. For example, the remote caregiver RC can perform a gesture that is recorded by the image capture device 218. The processing device 204 can then utilize the gesture recognition algorithms 208 to recognize gesture from the remote caregiver RC, and generate a control command for controlling the device in the patient environment PE.

As an illustrative example, the control command can cause the vital signs monitoring device 104 to display a different set of data based on a gesture of the remote caregiver RC recognized from the gesture recognition algorithms 208. As another illustrative example, the control command can cause an infusion pump to administer fluids and/or a medication based on a gesture of the remote caregiver RC recognized from the gesture recognition algorithms 208.

In some further examples, the remote caregiver RC can utter one or more commands that are recorded by the speaker and microphone unit 220. The speech recognition algorithms 210 can recognize speech from the remote caregiver RC, and then convert the speech into text to generate a control command for controlling a device in the patient environment PE such as the patient support apparatus 102, the vital signs monitoring device 104, and other devices.

In some examples, the remote caregiver telehealth device 200 can include additional optimizations for enhancing a telehealth consultation between the remote caregiver RC and the patient P. For example, the image capture device 218 can be optimized to automatically track the remote caregiver RC's position and/or location in the remote environment RE for framing a 3D image of the remote caregiver RC such that the 3D image is well contained within the field of view displayed on the image forming device 308 of the patient telehealth device 300.

Additionally, the virtual interface provided by the remote caregiver telehealth device 200 can allow the remote clinician RC to control the projector device 116 that is positioned inside the patient environment PE (see FIG. 1). As described above, the projector device 116 can display a holographic image 118 of the remote caregiver RC inside the patient environment PE. The holographic image 118 can improve engagement and interaction with the patient P.

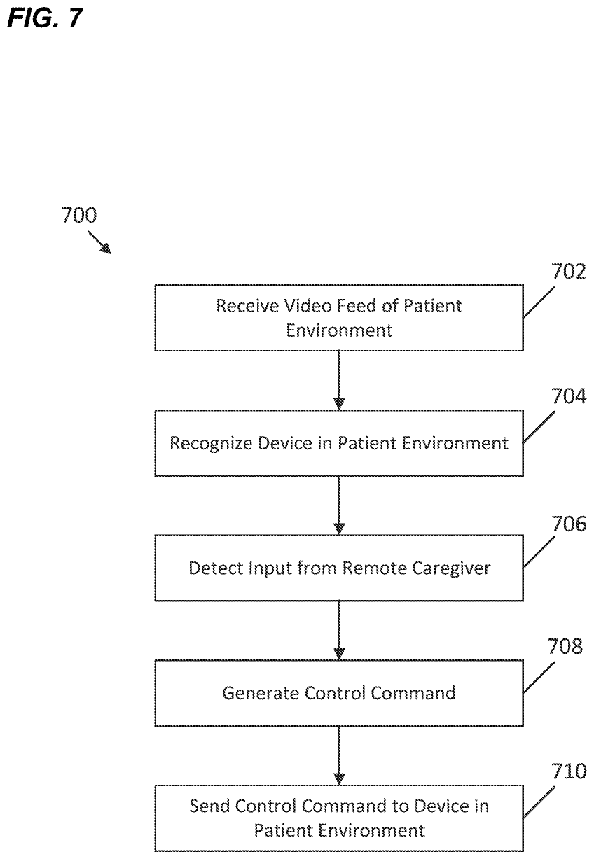

FIG. 7 schematically illustrates an example of a method 700 of conducting a telehealth consultation. The method 700 can be performed by the remote caregiver telehealth device 200. The method 700 includes an operation 702 of receiving a video feed of the patient environment PE. In some examples, the video feed is received by the camera 114 worn by the local caregiver LC. In other examples, the video feed is received by another device in the patient environment PE. In some examples, operation 702 can further include displaying the video feed such as on the one or more display devices 212 of the remote caregiver telehealth device 200.

Next, the method 700 includes an operation 704 of recognizing a device in the patient environment PE. The device can be recognized from the video feed received in operation 702. As an illustrative example, operation 704 can include recognizing the vital signs monitoring device 104 (see FIG. 1). In further examples, additional types of devices can be recognized.

Next, the method 700 includes an operation 706 of detecting an input of the remote caregiver RC directed to the device recognized in operation 704. In some examples, the input is a gesture such that operation 706 includes using the image capture device 218 to record the gesture from the remote caregiver RCE, and thereafter using the processing device 204 to perform the gesture recognition algorithms 208 to recognize the gesture from the remote caregiver RC.

In further examples, the input is a voice command such that operation 706 includes using the speaker and microphone unit 220 to record the voice command from the remote caregiver RCE, and thereafter using the processing device 204 to perform the speech recognition algorithms 210 to recognize the voice command from the remote caregiver RC.

Next, the method 700 includes an operation 708 of generating a control command based on the input detected in operation 706. In some examples, the control command is generated by using a lookup table that correlates control commands and gestures and voice commands. Additional techniques may be used to generate the control command.

Next, the method 700 includes an operation 710 of sending the control command generated in operation 708 to the device recognized in operation 704. The control command can be communicated from the remote caregiver telehealth device 200 to the device recognized in operation 704 via the communications network 110 (see FIG. 1).

The control command when received by the device recognized in operation 704 causes the device to perform an action. In examples where the device is the vital signs monitoring device 104, the control command can cause the vital signs monitoring device 104 to display a different set of data. As another example, the control command can cause the device to provide a therapy. For example, the control command can cause an infusion pump to administer fluids and/or a medication to the patient P based the input received from the remote caregiver.

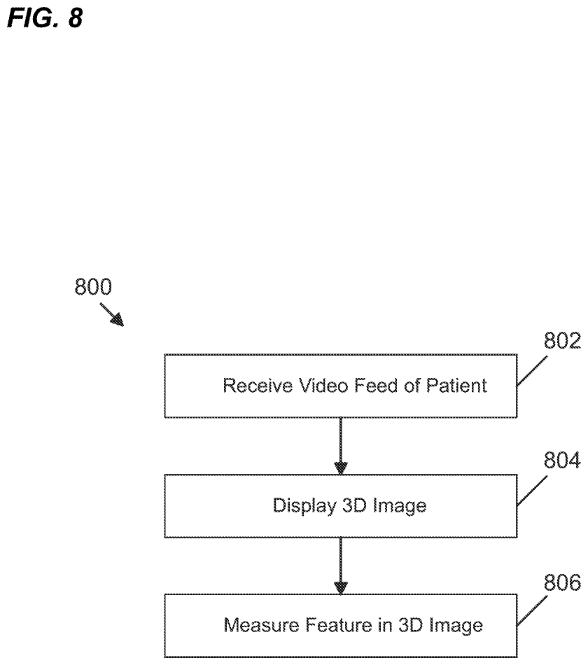

FIG. 8 schematically illustrates another example of a method 800 of conducting a telehealth consultation. In some examples, the method 800 is performed by the remote caregiver telehealth device 200. The method 800 includes an operation 802 of receiving a video feed of the patient P. The video feed is captured by the image capture device 314 of the patient telehealth device 300 and includes RGB-D data. The video feed is received by the remote caregiver telehealth device 200 via the communications network 110.

Next, the method 800 includes an operation 804 of displaying a 3D image based on the video feed received in operation 802. The video feed includes RGB-D data that allows the one or more display devices 212 to form a 3D image. For example, a display device such as an LCD display, and a lenticular lens can be used to generate the 3D image. When the 3D image is displayed on the image forming device 214, the 3D image can be viewed by the remote caregiver RC without requiring specialized eyewear accessories such as goggles, eyeglasses, and the like.

Next, the method 800 includes an operation 806 of measuring a feature in the 3D image displayed in operation 804. For example, operation 806 can include measuring a size and depth of a wound based on the RGB-D data of the video feed. Advantageously, the feature can be measured in operation 806 without having to perform complex spatial calibrations, photometric interpretation, and other imaging techniques that rely on pixel counts.

The various embodiments described above are provided by way of illustration only and should not be construed to be limiting in any way. Various modifications can be made to the embodiments described above without departing from the true spirit and scope of the disclosure.

What is claimed is:

1. A device for conducting a telehealth consultation, the device comprising:
at least one processing device; and
a memory device storing instructions which, when executed by the at least one processing device, cause the at least one processing device to:
receive a video feed of a patient environment;
recognize a local device in the video feed of the patient environment;
detect an input directed to the local device;
generate a control command based on the input; and
send the control command to the local device, wherein the control command causes the local device to perform an action during the telehealth consultation.

2. The device of claim 1, wherein the memory device stores further instructions which, when executed by the at least one processing device, cause the at least one processing device to:
perform a gesture recognition algorithm to recognize the input as a gesture.

3. The device of claim 1, wherein the memory device stores further instructions which, when executed by the at least one processing device, cause the at least one processing device to:
perform a speech recognition algorithm to recognize the input as a voice command.

4. The device of claim 1, wherein the control command causes the local device to change a data display.

5. The device of claim 1, wherein the control command causes the local device to provide a therapy to the patient.

6. The device of claim 1, wherein the memory device stores further instructions which, when executed by the at least one processing device, cause the at least one processing device to:
display the video feed of the patient environment; and
augment the video feed to include a display of clinical data measured by the local device.

7. The device of claim 6, wherein the video feed is displayed in three dimensions.

8. A method of conducting a telehealth consultation, the method comprising:
receiving a video feed of a patient environment;
recognizing a local device in the video feed of the patient environment;
detecting an input directed to the local device, the input detected from a remote caregiver watching the video feed;
generating a control command based on the input; and
sending the control command to the local device, wherein the control command causes the local device to perform an action during the telehealth consultation.

9. The method of claim 8, further comprising:
displaying the video feed of the patient environment; and
augmenting the video feed to include a display of clinical data measured by the local device recognized in the video feed.

10. The method of claim 9, wherein the video feed is displayed in three dimensions.

11. The method of claim 8, further comprising:
performing gesture recognition algorithms to recognize the input.

12. The method of claim 8, further comprising:
performing speech recognition algorithms to recognize the input.

13. A device for conducting a telehealth consultation, the device comprising:
at least one processing device; and
a memory device storing instructions which, when executed by the at least one processing device, cause the at least one processing device to:
receive a video feed of a patient;
display a three-dimensional image based on the video feed;
determine a measurement of a feature in the three-dimensional image; and
display the measurement in the three-dimensional image.

14. The device of claim 13, further comprising:
an image forming device, the image forming device including:
a display device; and
a lenticular lens.

15. The device of claim 13, further comprising:
an image capture device configured to capture an image of a user.

16. The device of claim 15, further comprising:
a mirror covering the image capture device, the mirror allowing the image capture device to capture images without disturbing the three-dimensional image.

17. The device of claim 13, wherein the video feed includes red, green, blue, and depth (RGB-D) data, and the three-dimensional image is generated using the RGB-D data.

18. The device of claim 17, wherein the measurement is determined based on the RGB-D data.

19. The device of claim 13, wherein the measure is of a wound size.

20. The device of claim 13, wherein the memory device stores further instructions which, when executed by the at least one processing device, cause the at least one processing device to:
recognize an object in the three-dimensional image;
augment the three-dimensional image to include a display of clinical data associated with the object recognized in the three-dimensional image.
